(19) United States
(12) Patent Application Publication
(10) Pub. No.: US 2021/0000907 A1
(43) Pub. Date: Jan. 7, 2021

(54) MICRONEEDLE-MEDIATED DELIVERY OF TOLEROGENIC IMMUNOTHERAPEUTICS

(71) Applicant: University of Maryland, College Park, College Park, MD (US)

(72) Inventors: Christopher M. JEWELL, Silver Spring, MD (US); Lisa H. TOSTANOSKI, Washington, DC (US); Emily GOSSELIN, Hudson, NH (US)

(21) Appl. No.: 16/492,731

(22) PCT Filed: Mar. 15, 2018
(86) PCT No.: PCT/US18/22549

§ 371 Date: Sep. 10, 2019

Related U.S. Application Data
(60) Provisional application No. 62/471,807, filed on Mar. 15, 2017.

(51) Int. Cl. A61K 38/02; A61K 9/00; A61P 25/28

(52) U.S. Cl. CPC A61K 38/02; A61P 25/28; A61K 9/0021

(57) ABSTRACT

Methods, compositions and kits are provided that include microneedles coated with or formed of antigens to which immune tolerance is desired. Use of the microneedles is demonstrated using glatiramer acetate and animal models of multiple sclerosis. Dose sparing and beneficial polarization of immune responses are demonstrated.

### CROSS-REFERENCE TO RELATED APPLICATIONS

This application claims priority to U.S. Provisional Patent Application No. 62/471,807, filed Mar. 15, 2017, the disclosure of which is incorporated herein by reference.

### BACKGROUND OF THE DISCLOSURE

Multiple sclerosis (MS) occurs when myelin in the central nervous system (CNS) is mistakenly attacked by self-reactive T cells (e.g., T_{H}17, T_{H}1) and antibodies, causing inflammation and loss of CNS function. This attack creates slow, debilitating progression that is plaques patients and their families both in quality of life and financially. Although existing treatments for MS have been beneficial, there are no cures and lack of specificity can leave patients immunocompromised. New delivery technologies that have the potential to provide knowledge for the field, and new freedom to patients with poor mobility or limited dexterity are needed. For example, the ability of MS patients to self-administer in-use injectable prescriptions would greatly reduce reliance on frequent transportation to the clinic, or on loved ones and healthcare workers just to receive simple injections. The present disclosure is pertinent to these and other needs.

### SUMMARY OF THE DISCLOSURE

The present disclosure relates to methods, compositions and kits that include microneedles (MNs), and uses thereof for promoting immune tolerance. The MNs can be coated with, or partially or fully made of antigens to which immune tolerance is desired. In embodiments, the disclosure accordingly provides methods for promoting immune tolerance in an individual comprising intradermal administration of an effective amount of one or more antigens to which immune tolerance is desired using an array of MNs. The immune tolerance is increased and/or improved relative to a control value. The disclosure is demonstrated using glatiramer acetate as an antigen, and dose sparing and beneficial polarization of immune responses relative to injected antigens at higher doses is shown. In embodiments, a composition comprising the antigen, such as glatiramer acetate, is coated on the surface of the MNs. In embodiments, the MNs are formed of a composition comprising, consisting essentially of, or consisting of the antigen(s). In an embodiment, the antigen, such as glatiramer acetate, persists in the skin of the individual for a period of 1, 2 or 3 three days, which may occur after the MNs dissolve. In embodiments, the MNs comprise ≤2.0 mg of the glatiramer acetate per MN array administration. In embodiments, the MNs comprise not more than 0.2 mg of the glatiramer acetate, such as not more than 0.2 mg of the glatiramer acetate in a single intact MN array.

In embodiments, use of the MNs as described herein results in immune tolerance that comprises any of: i) an increase in regulatory T cells (TREGs) in any of: lymph nodes, spleen or the central nervous system of the individual; and/or ii) a decrease of inflammatory lymphocytes in any of: lymph nodes, spleen or the central nervous system of the individual. In an embodiment, an increase in TREGS occurs in cervical lymph nodes of the individual. In an embodiment, a decrease of inflammatory lymphocytes occurs. The decrease can be of inflammatory lymphocytes that are CD45⁺CD4⁺ T cells.

In certain approaches, the MNs are used to treat an individual who has Multiple sclerosis (MS), which can include but is not necessarily limited to primary-progressive multiple sclerosis (PPMS), relapsing-remitting MS (RRMS), secondary-progressive MS (SPMS), or progressive-relapsing MS (PRMS). In embodiments, as a consequence of use of MNs as described herein the individual who has MS exhibits an inhibition of onset of paralysis, or a reduction in paralysis. In an embodiment, the MN arrays of this disclosure are self-applied by the individual in need of the immune tolerance.

### BRIEF DESCRIPTION OF THE FIGURES

FIG. 1. Images showing microneedles (MNs) prepared with labeled glatiramer acetate (GA) lead to panel A) stable MNs with panels B, C and D) fibrous surfaces visible by scanning electron microscopy (SEM; panel C). Labeled GA is observed as diffuse signal throughout each MN (panel E).

FIG. 2. Images showing: panel A) MNs penetrate skin in ears of live mice, panel B) resulting in observable change in height of MNs, panels C, D and E) Painting ears with trypan blue after MN removal confirms penetration of dermis. Panel F) GA-MNs deliver GA to skin, persisting for at least 3 days.

FIG. 3. Plots showing: Panel A) Daily GA dosing drives dose-dependent effects. Panel B) GA-MNs improve disease progression and (panel C) maintenance of weight during Experimental Autoimmune Encephalomyelitis (EAE). During relapsing-remitting MS (RR-EAE), GA-MNs improve (panel D) disease progression and (panel E) mean score at doses that injectable GA does not.

FIG. 4. Graphical summaries of data showing: GA-MNs increase the number of (panel A) lymphocytes and (panel B) T_{REG} in cervical lymph nodes (LNs), but do not impact (panel C) lymphocyte counts in the spleen

FIG. 5. Graphical summaries of data showing: GA-MNs (panel A) reduce the number of lymphocytes in the spinal cord, and (panels B and C) decrease the number of CD45⁺CD4⁺ T cells in these tissues.

### DESCRIPTION OF THE DISCLOSURE

The present disclosure is related to inducing immune tolerance in individuals who have autoimmune disorders, and is exemplified via compositions and methods that are shown to be surprisingly efficacious in reducing and even reversing symptoms of MS in animal models, among other tolerogenic effects on the immune system.

Throughout this specification, where a value of ranges is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges which may independently be included in the smaller ranges is also encompassed within the disclosure, subject to any specifically excluded limit in the stated range. All dose amounts and ranges of dose amounts, and dosing schedules described herein are expressly included by this disclosure.

In embodiments, the disclosure relates to promoting immune tolerance in an individual in need thereof. The disclosure facilitates promoting therapeutic and/or prophylactic immune tolerance using lower dosing of antigens than has been previously possible. Tolerance is produced via intradermal administration of an effective amount of one or more antigens to the individual using one or more arrays of MNs. The disclosure in various implementations thus excludes subcutaneous and intravenous injections. Accordingly, the disclosure can exclude any needle-based approach wherein the needle accesses subcutaneous tissue. In embodiments, the disclosure therefore provides for an increase in tolerance relative to a control value for immune tolerance produced by injection, such as a subcutaneous or intravenous injection of the same antigen type, which may be administered in a dosage that is higher than the dosage administered using the MNs as described herein. Further, and without intending to be bound by any particular theory, it is considered that compared with injection or even topical application, MNs of this disclosure will offer other numerous advantages, such as ease of application, elimination of pain (owing to needles that are too short to reach pain receptors), elimination of medical sharps, and improved stability of biomacromolecular cargos. This latter can mean the present MN formulations will not need refrigeration. Thus, in certain aspects, the disclosure provides MN formulations that are shelf-stable without the need to be held in reduced temperature and can be kept without refrigeration for a period of at least three days without losing efficacy in promoting immune tolerance. Further, it is considered that embodiments of this disclosure may improve quality of life for autoimmune disease patients, such as multiple sclerosis patients. In this regard, the inability to self-inject or to drive to a clinic creates substantial obstacles for MS and other autoimmune disease patients, both in burden and cost. Transportation to a clinic or frequent need for assistance to administer medications are real considerations, but many studies also reveal the critical impact these challenges create to for compliance, decreasing both efficacy and patient quality of life. These aspects highlight certain benefits of the present disclosure, such as less frequent drug administration relative to injectable MS therapies. Accordingly, the disclosure enables drugs to be self-administered by reducing the coordination threshold, while improving drug effectiveness by increasing compliance.

In embodiments, MN administration according to this disclosure is such that antigens reach skin-resident immune cells, including but not necessarily limited to antigen presenting cells (APCs), such as Dendritic cells (DCs), including but not limited to plasmacytoid (pDCs), and/or Langerhans cells. In this regard, and as known in the art, APCs are concentrated in the skin, playing an important role in promoting peripheral tolerance during autoimmunity and maintenance of homeostasis following pro-immune responses. DCs and pDCs in particular, are important in these processes and can also help promote TREGS. For example, pDCs from patients treated with interferon-β exhibit reduced inflammatory and T_{H}1/T_{H}17-polarizing cytokines. These abilities are enabled by display of regulatory ligands (e.g., PD-1) and secretion of tolerizing cytokines (e.g., IL-10, TGF-β) that limit T cell proliferation and polarize cells that do divide toward T_{REG}. In certain embodiments, the present disclosure demonstrates, using lower dosages of a representative antigen formulation (glatiramer acetate (GA), discussed further below) delivered by MNs, an increase in TREGS in lymph nodes, spleen and the central nervous system in an animal model of MS, as further described below. In embodiments, increased TREGs are present in cervical lymph nodes.

In embodiments the disclosure also demonstrates a decrease of inflammatory lymphocytes in lymph nodes, spleen or the central nervous system of the animal model using the same MN-based approach, as further described below. Thus, the disclosure demonstrates increases in regulatory cells/protective cells and decreases in inflammatory/effector cells as a result of the MN-based delivery of antigen. In embodiments, an increase in regulatory lymphocytes is not accompanied by an increase in total lymphocytes, i.e., TREGS are increased, but inflammatory lymphocytes are not. Non-limiting examples of inflammatory cells include CD45⁺CD4⁺ T cells. In embodiments, one or effects of performing a method of this disclosure can be ascertained by measuring to determine, for example, T cell proliferation, phenotype, and cytokine levels, such as by using fluorescence dilution (CSFE or Efluor 670), FACS, and ELISA assays.

In embodiments, one or more effects of performing a method of this disclosure can be ascertained by determining that T cells proliferate—indicating the intradermally delivered antigen has been processed and presented. Values that can be analyzed include but are not limited to: T cell phenotypes, such as T_{REG} (CD4+/CD25+/Foxp3+; increased TGF-β, IL-10), T_{H}17 (CD4+/RORγ+; increased IL-17, IL-23, IFN-γ), T_{H}1 (CD4+/T-bet+; increased IFN-γ, TNF), and T_{H}2 (CD4+/GATA3+; increased IL-4, IL-6). Relevant regulatory and inflammatory signals for dendritic cells include but are not necessarily limited to activation markers (i.e., high levels of CD40, CD80, CD86, MHCII), along with, for example, ELISA assays to detect inflammatory (e.g., GM-CSF, IL-1β, TNFα) and regulatory cytokines (e.g., IL-10, TGF-β). Likewise, patient samples can be analyzed to determine, for example, whether peripheral blood mononuclear cells (PBMCs) isolated from human patients treated with MNs of this disclosure can recognize myelin antigens that are released from the MNs. For example, PBMC supernatants can be analyzed for inflammatory and regulatory cytokines. The cytokine panel can include, but is not necessarily limited to IL-1α, IL-2, IL-6, IL-10, IFN-γ, TNF-α, TGF-β and IL-4.

By way of the present figures and descriptions thereof, the disclosure provides representative and non-limiting demonstrations of MN and array dimensions that are suitable for use in promoting immune tolerance using the methods described herein. In embodiments, suitable MN are used, in conjunction with appropriate application of force to skin of an individual, such that the MNs do not fully pierce through the dermis. Those skilled in the art will be able to determine from this disclosure suitable MN dimensions, and dimensions of arrays with which the MNs are physically associated.

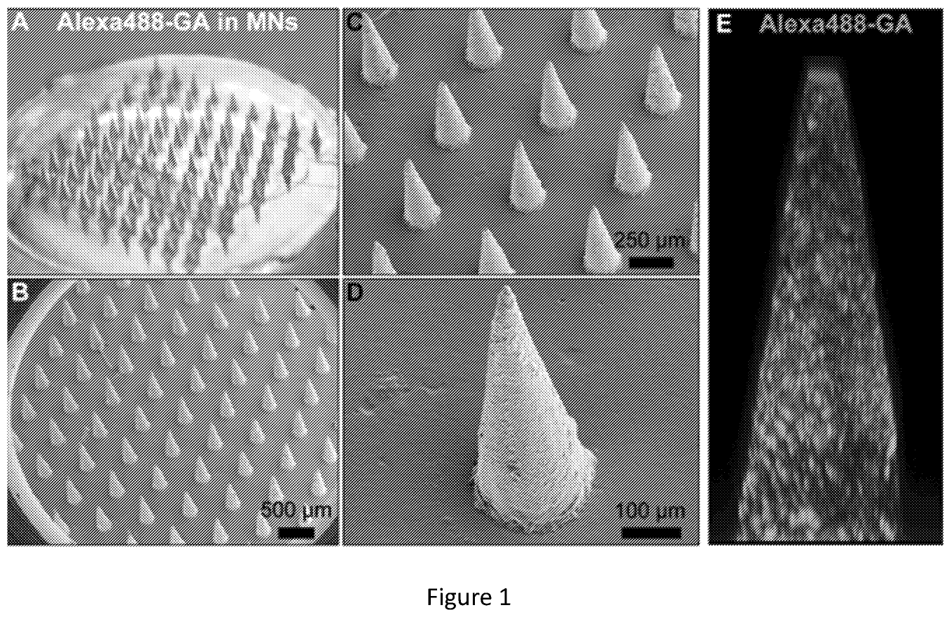

MN arrays useful for practicing the present disclosure can have a variety of configurations. Hollow and solid MNs are included. Non-limiting examples of MN dimensions and arrays are provided in FIG. 1, described further below. The array includes a plurality of MNs positioned on a MN substrate. Each MN has a height, which is the length from the tip of the MN to the MN base at the substrate. In some embodiments, including any one of the embodiments described herein, each of the plurality of MNs (or the average of all of the plurality of MNs) has a height of approximately 600 μm-650 μm. In a non-limiting aspect ratio, each MN, or the average of the MNs, has a height of 650 um and a 250 um bottom diameter at the substrate.

In embodiments a single array comprises 80-1500 MNs. In some embodiments, the array of MNs comprises 200 to 1500 per cm² MNs. A single MN or the plurality of MNs in a MN array can also be characterized by an aspect ratio. The aspect ratio of a MN is the ratio of the height of the MN to the width (at the base of the MN). The aspect ratio can be presented as height:width relationship. In a non-limiting aspect ratio, each MN, or the average of the MNs in the array, has a height of 650 um and a 250 um bottom diameter at the substrate.

MNs in an array of this disclosure can have a variety of shapes. In some embodiments, an MN has a square pyramidal shape or the shape of a hypodermic needle. In some of these embodiments, the shape is square pyramidal.

In certain approaches, embodiments of MN arrays can be made using molds formed of any suitable substrate, one non-limiting example of which is poly(dimethylsiloxane) (PDMS). In an embodiment, the molds have 600 μm geometries. Each GA (or other antigen as described herein) can be stabilized with mannitol (or sucrose if higher viscosities are required) by, for example, systematically varying the excipient concentration from 0-15% to achieve a Young modulus of at least 10.0 kPA; without intending to be constrained by any particular parameter, it is considered that this the value needed to adequately penetrate skin. A suitable solution, such as poly(lactic acid) (PLA) solution—can be used to create a non-dissolvable backing to be applied in the top of each well. The arrays can be treated, such as by being lyophilized for a suitable period, such as for 24 hrs, then demolded. The modulus of the MNs can be measured for example, by a Dynamic Mechanical Analysis (DMA) system equipped with a plate tool to create a storage and loss curve. To analyze dosing control, similar analysis can be made in which the input GA (or peptide dose) is varied from 100-1000 μg. Although the Examples below demonstrated efficacy, it is expected that loading can be increased between 500-1000 μg/MN patch (vs. the current 200 μg/MN patch used in the Examples). Loading can be measured by dissolving the MN in sterile H₂O for a period of time, such as for 30 min. and measuring the antigen concentration using any suitable approach, such as by microBCA. Labeled GA or peptide can be used to allow direct visualization for imaging, cell, or biodistribution studies. Labeling can be performed using any suitable approach, some of which are demonstrated in the Examples of figures of this disclosure. Properties of MN during dissolution can be measured and the distribution of colloidal (peptide) diameters, surface charge, and solution polarization (e.g., level of tertiary and peptide interactions) of the dissolving MNs using dynamic light scattering, zeta potential, and circular dichroism, respectively, can be determined. Using such techniques, when provided the benefit of the present disclosure, those skilled in the art can assess the values for each parameter for GA released from MNs. Such measurements can be performed by incubating distinct MNs for increasing durations (such as by using 30 second increases until 10 minutes) in water, then measuring the modulus of the MN to determine the time to reach failure (i.e., total dissolution), and collecting the incubation solutions for the analysis types as described. A second fraction of each release solution can be used for cell culture analysis.

In some embodiments, an array of MNs as described herein may be in the form of a patch, which may comprise a combination of an MN array, pressure sensitive adhesive, and backing. Suitable pressure sensitive materials and adhesives are known in the art. The MNs may be arranged in any desired pattern or distributed over the MN substrate randomly.

All potential combinations of distinct types of MNs described herein are encompassed by the disclosure. All methods of making the microneedles and MN arrays described herein are included in this disclosure.

The MNs can be coated with one or more antigens, or can be formed by the antigens themselves. The MNs themselves may comprise, consist essentially of, or consist of the antigens. In embodiments, the MNs are formed solely from one, or more than one antigen, and one, or more than one additive, including but not necessarily limited to pharmaceutically acceptable additives, such as excipients, stabilizers, and the like. In embodiments, the MNs comprise one or more antigens and one or more stabilizers that are soluble in an aqueous solution, including but not necessarily limited to carbohydrates such as sucrose, or sugar alcohols. Non-limiting examples of suitable sugar alcohols include mannitol and sorbitol.

MN arrays can be applied to any suitable location of the skin of an individual, and can be maintained and replaced according to schedules that will be apparent to those skilled in the art, given the benefit of this disclosure. In embodiments, the MNs may persist in the skin of the individual, or may dissolve. Dissolution of the MN structure can proceed rapidly. In embodiments, the MNs are dissolved within a period of from 1-30 minutes, such as 1-10 minutes. In embodiments, the patch comprising the substrate is removed subsequent to the dissolution of the MNs.

In certain approaches, when applied to an individual, the one or more antigens can be the only components of the MNs that promote immune tolerance, and/or are the only components of the MNs. In embodiments, trace amounts of other materials used in forming the MNs may be present. In embodiments, MS arrays of this disclosure do not comprise any amino acids that are not part of the antigens. In embodiments, the MS arrays do not comprise histidine, such as histidine that is not a component of an antigen.

The antigens comprised by the MNs are characterized as those against which suppression of an immune response is desirable, such as self-antigens. Embodiments of the disclosure are illustrated using MNs that are coated with, and also formed at least in part, GA. This highly successful first-line MS drug is a random co-polymer of amino acids that occur at high levels in myelin and has typically been injected subcutaneously using daily or 3×/weekly intervals (Aharoni, R. The mechanism of action of glatiramer acetate in multiple sclerosis and beyond. Autoimmun Rev 12, 543-553, doi:DOI 10.1016/j.autrev.2012.09.005 (2013)). It is commercially available, and is sold for example, under the tradename COPAXONE® by TEVA® and is available generically from, for example, MYLAN®. In an embodiment, a formulation of GA used in a method of this disclosure comprises or consists of the acetate salts of synthetic polypeptides, containing four naturally occurring amino acids: L-glutamic acid, L-alanine, L-tyrosine, and L-lysine with an average molar fraction of 0.141, 0.427, 0.095, and 0.338, respectively. GA is also described under PUBCHEM compound ID CID 3081884, the description of which is incorporated herein by reference. Its structural formula is C₂₅H₄₅N₅O₁₃ and its molecular weight is 623.657 g/mol.

In embodiments, the disclosure comprises promoting immune tolerance in an individual via intradermal administration of an effective amount of glatiramer acetate to the individual using an array of MNs. The administration is such that the immune tolerance is increased relative to a control value for immune tolerance produced by injection of glatiramer acetate. In embodiments, glatiramer acetate is coated on the surface of the MNs, or the MNs are formed by a composition comprising the glatiramer acetate.

In one embodiment, the glatiramer acetate persists in the skin of the individual for a period of at least three days, subsequent to the application of the MN array.

In embodiments, the total amount of glatiramer acetate as an effective amount in an intact MN array of this disclosure is less than the recommended GA dose for an MS patient at the time of the filing of this application or patent. In embodiments, the dose is 0.9, 0.8, 0.7, 0.6, 0.5, 0.4, 0.3, 0.2 or 0.1 times the recommended dose. In embodiments, the recommended dose is between 20-40 mgs of GA per day. In embodiments, the dose is administered less than daily, such as once weekly, or twice weekly, or three times a week.

In embodiments, an effective amount of GA to promote immune tolerance is less than less than 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0 mgs, inclusive, and including all numbers to the second decimal point there between. In embodiments an effective GA dose provided by MNs of this disclosure is from 0.01-5.0 mg, inclusive, and including all numbers to the second decimal point there between. In embodiments, the amount of glatiramer acetate comprise ≤2.0 mg of the glatiramer acetate per MN array administration. In embodiments, the amount of glatiramer acetate comprise from 5 μg-200 μg. In embodiments, the amount of glatiramer acetate comprises no more than 200 μg. In embodiments, only a single array, i.e., a single patch, is applied to an individual at a time. A single patch can be considered a single dose.

Control values can be established using any of these parameters. In embodiments, at least one, or only one MN array is applied per day. In embodiments, a lower dose of MN-applied GA according to this disclosure is a least as effective in promoting immune tolerance as an injected dose of the same amount, or an injected dose of a larger amount. In embodiments, a lower dose of GA administered using an MA array as described herein is 1.5-10× as effective as the same or a higher dose of injected MA. In certain embodiments, the MN-approach is more effective in increasing TREGS in any of the lymph nodes, spleen or the central nervous system in the individual than the injected control value. The amount and location of T_{REGS} in any particular location in an individual can be determined using standard approaches, non-limiting demonstrations of which are described in the Examples below. In embodiments, any indicator of immune tolerance, including but not limited to symptom scoring, cellular profiles, and cellular and/or molecular indicators of inflammation can last beyond cessation of treatment, for a period of 1, 2, 3, 4, 5, 6, 7 days, or at least one week, or at least two weeks, or more than two weeks.

In addition to GA, the disclosure includes MNs comprising or formed from polyelectrolyte multilayer (PEM) materials that can be built entirely from immune signals. These immune-PEMs (iPEMs) provide a platform for rationally-designing PEM coatings from immune signals in a way that reduces or eliminates potentially confounding intrinsic properties of synthetic polymers or other structural components often included in PEM films. Suitable examples of iPEMs are described in U.S. Patent Publication No. 20180028646, the entire disclosure of which is incorporated herein by reference. In embodiments, in addition to or instead of GA, the antigen delivered using MNs as described herein can be any antigen that is positively correlated with MS.

In embodiments, the individual that is treated with one or more MN arrays of this disclosure has been diagnosed with or is suspected of having MS, and thus can include individuals who have one or a combination of primary-progressive multiple sclerosis (PPMS), relapsing-remitting MS (RRMS), secondary-progressive MS (SPMS), or progressive-relapsing MS (PRMS). Accordingly, in certain embodiments the disclosure is suitable to promote myelin-specific tolerance. In certain embodiments, the individual has been diagnosed with or is suspected of having PPMS. In this regard, and as is known in the art, PPMS is generally characterized by persistent worsening of neurologic function, but without separate relapses or periods of remission. PPMS thus differs from relapsing forms of MS in that the relapsing forms comprise at least two separate locations of damage in the central nervous system (dissemination in space) that occurred at different time points (dissemination in time). The inflammatory events that result in this damage comprise the relapses (sometimes referred to alternatively as attacks or exacerbations). In contrast, PPMS comprises a gradual change in functional abilities over time. Accordingly, because PPMS and relapsing MS are considered distinct disorders (but are not necessarily mutually exclusive in any particular individual) they have different diagnostic criteria. Specifically, PPMS can be diagnosed based on a combination of criteria that comprises a) at least one year of disease progression, which typically includes worsening of neurological function without remission, and b) at least two of: i) a type of lesion in the brain that is recognized by a medical professional skilled in the art of MS diagnosis; ii) two or more lesions of a similar type in the spinal cord; and iii) evidence in the spinal fluid of a oligoclonal band of immunoglobulins, and/or an elevated IgG index, which are both signs of immune system activity in the central nervous system. In certain embodiments of this disclosure, the individual has been diagnosed with or is suspected of having PPMS. In embodiments, PPMS may be the only type of MS that the individual is suspected of having, or has been diagnosed with. In certain embodiments, a method of this disclosure results in a slowing of the progression of symptoms of PPMS, and can even include a reversal of PPMS progression. In connection with this and as known in the art, common symptoms of MS, which can be encompassed by PPMS, include but are not necessarily limited to fatigue, walking difficulties, spasticity, dizziness and vertigo, blurred vision and pain upon eye movement, bladder and bowel dysfunction, numbness or tingling, sexual dysfunction, pain, and cognitive changes, such as complications in the ability to learn and remember information, problem solving and the like. Less common symptoms include but are not necessarily limited to difficulties with speech or swallowing, tremors, seizures and breathing problems. In embodiments, the present disclosure comprises a method of inhibiting the progression of the severity of one or more of these or other MS symptoms in an MS patient, such as a PPMS patient. In embodiments, inhibiting or reducing a symptom means the severity of the symptom is lessened, and/or the rate at which the symptom progresses is slowed, and/or the symptom is prevented from manifesting, and/or the symptom is eliminated. In one embodiment, the instant disclosure includes a demonstration of reducing MS symptoms, and using a common and well-characterized model of progressive MS, Experimental Autoimmune Encephalomyelitis (EAE).

As an alternative, or in addition to GA, any other myelin antigen(s) can be used in embodiments of this disclosure, provided the myelin antigen is recognized in whole or in part by a component of the immune system of the individual in need of treatment. Those skilled in the art will recognize that myelin is synthesized by different cell types, and can vary in composition and structure, but is defined as the material that makes up the so-called sheath of myelinated axons in vertebrates. Myelin in its form in myelinated axons comprises about 40% water; its dry mass is approximately 70-85% lipids and 15-30% proteins. In general, and without intending to be limited by any particular theory, it is considered that any of the lipids or proteins or fragments thereof that are inappropriately recognized by the immune system of an individual in need of treatment can function as a suitable antigen in the compositions and methods of the present disclosure. In embodiments, the myelin antigen comprises a lipid or immunogenic fragment thereof, exemplary lipids including but not necessarily limited to galactocerebroside and sphingomyelin. In embodiments, the myelin antigen comprises a protein or immunogenic fragment thereof, exemplary proteins including but not necessarily limited to myelin basic protein, myelin oligodendrocyte glycoprotein (MOG), and proteolipid protein. Without intending to be constrained by any particular theory, immunogenic fragments are considered to be those that are recognized by the immune system of an individual who has MS. In embodiments, the present disclosure is considered to result in induction of immune tolerance to such antigens. In embodiments, the antigen comprises or consists of myelin, a peptide fragment thereof, or a combination or peptide fragments. In embodiments, the antigen can be any of myelin peptide fragment MOG1-20, MBP13-32, MOG-35-55; myelin basic protein MBP13-32, MBP83-99, MBP84-104, MBP111-129, MBP146-170, or proteolipoprotein PLP139-151, PLP139-154, PLP178-191.

Embodiments of the disclosure can include co-administration, or including in the MNs, tolerogenic agents, such as any agents that function as an inhibitor of the mammalian target of rapamycin (mTOR), also known as FK506-binding protein 12-rapamycin-associated protein 1 (FRAP1). In embodiments, the mTOR inhibitor is rapamycin, or a rapalog. In embodiments, the mTOR inhibitor comprises Sirolimus, Temsirolimus, Everolimus, Deforolimus, or a second generation mTOR inhibitor generally known to function as an ATP-competitive mTOR kinase inhibitors, and/or mTORC1/mTORC2dual inhibitors. In embodiments, the tolerogenic agent comprises a cytokine or a chemokine or a growth factor or an interferon or a transcription factor, or other small molecule drugs that may include but are not limited to retinoic acid or mycophenolic acid. In embodiments a combination of such tolerogenic agents can be used.

Due at least in part to reductions in dosing, the disclosure unexpectedly reduces severity of at least one symptom of MS. In certain embodiments, a reduction in the severity of MS symptoms in, for example, PPMS patients comprises a slowing of the progression of PPMS, a cessation of the progression of the PPMS, or elimination of at least one symptom of the PPMS. This MS approach can also produce a systemic reduction of inflammation in the individual.

Accordingly, the present disclosure provides a therapeutically feasible approach for not only reducing, but stopping and even reversing progression of symptoms of MS, including PPMS.

In embodiments, the disclosure comprises kits for prophylaxis and/or therapy for an autoimmune disease, such as MS. The kits can comprise any of the MNs described herein. The kits can comprise printed material, such as instructions for application and removal of a MN array, and/or an indication that the kit is for use in treating, for example, an MS patient.

The following Examples are intended to illustrate but not limit the disclosure.

### Example 1

This Example demonstrates a non-limiting method for making representative MNs. In particular, to determine if GA could be loaded in MNs, we first used N-hydroxysuccinimide (NHS) ester chemistry and dialysis against mannitol (4%) to label and purify undiluted, liquid GA (COPAXONE®, Teva) with a fluorescent dye (Alexa-488). We prepared poly(dimethylsiloxane) (PDMS) molds by lithography and loaded the molds with the labeled GA, along with defined concentrations of sucrose or mannitol to arrive at a stable, but H₂0-soluble formulation. A solution of non-H₂0 soluble solution of poly(lactic acid) (PLA) was then added to the top of the array to form a non-dissolvable backing. The loaded molds were then lyophilized (i.e., dried) under near-complete vacuum and subsequently cooled. Demolding the MN patches resulted in stable patches with a visible yellow color (FIG. 1, panel A), compared with GA-MNs prepared with unlabeled GA. Inspection of GA-MNs by scanning electron microscopy (SEM) revealed uniformly-spaced MNs, each with fibrous peptide surfaces attached to the smooth PLA back (FIG. 1, panels B-D). Confocal microscopy confirmed labeled GA localized within individual MNs at wavelengths corresponding to Alexa-488 (FIG. 1, panel E).

### Example 2

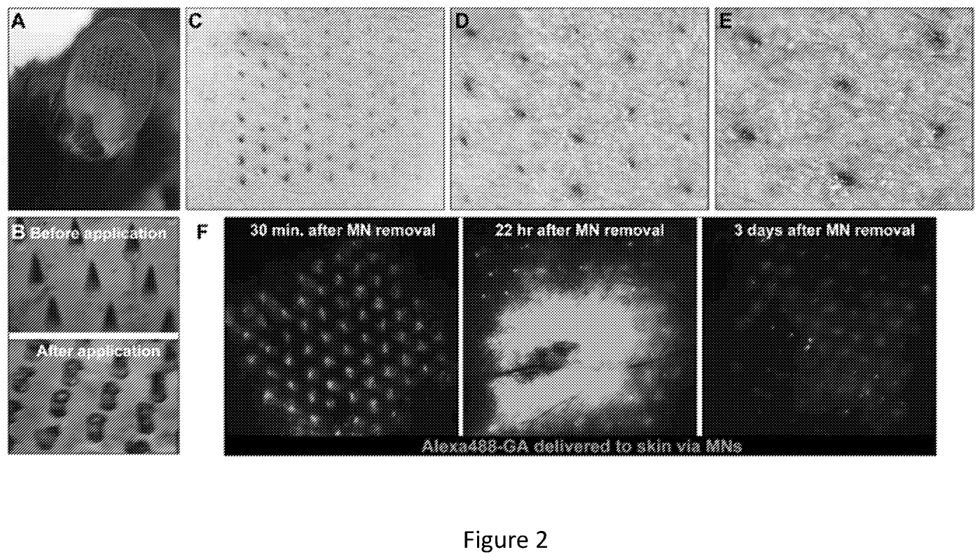

This Example demonstrates that GA-MNs quickly deliver GA to live mice and persists at application site. To provide this demonstration, we formulated MNs with trypan blue—a dye that confirms penetration of the dermis by blue staining of cells. Application of the MN to the ears of live mice for 5 min. resulted in an array pattern on the ear (FIG. 2, panel A) and a dramatic decrease in MN height visualized by photographic imaging of the MN before and after application (FIG. 2, panel B). In a similar approach, MNs without trypan blue were applied to the ear, then subsequently, ears were painted with trypan blue, confirming clear penetration of the dermis in a uniform pattern (FIG. 2, panels C-E). Next we formulated GA-MNs using Alexa488 GA and applied the GA-MNs to the ears of live mice for 5 min. The MNs were removed, then the mice were euthanized at specific time points to assess the delivery of GA to the skin and the persistence at the application site. Immunofluorescence revealed a grid pattern 30 min. after removal of the MN that reached a bright, less defined pattern 22 hours after MN removal, and a return to a dimmer, diffuse array pattern 3 days after removal (FIG. 2, panel F). Thus, GA-MNs deliver GA to living mice and maintain GA at the MN application site for at least 3 days, which is longer than the intervals between the current 3×/weekly GA dose presently used in human MS patients, i.e., a dose about every 2.33 days.

### Example 3

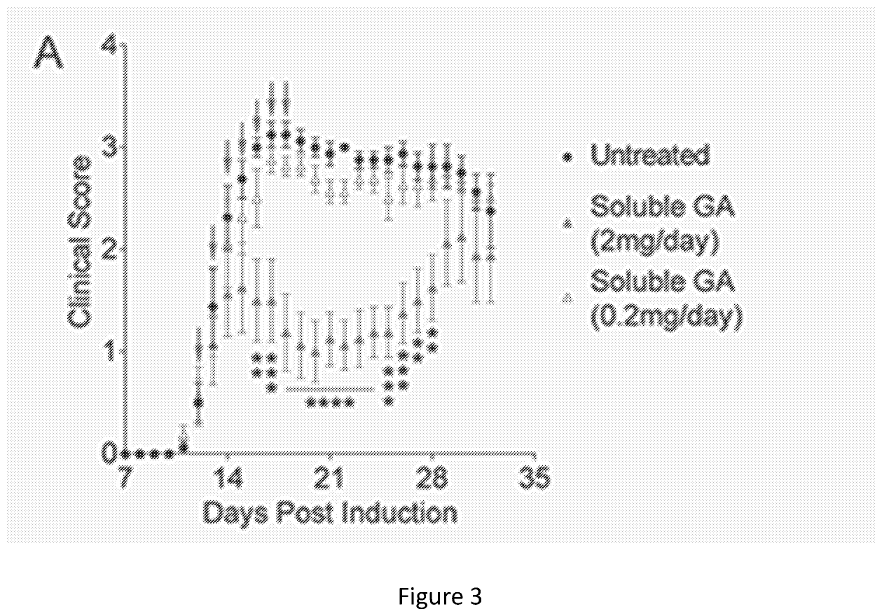

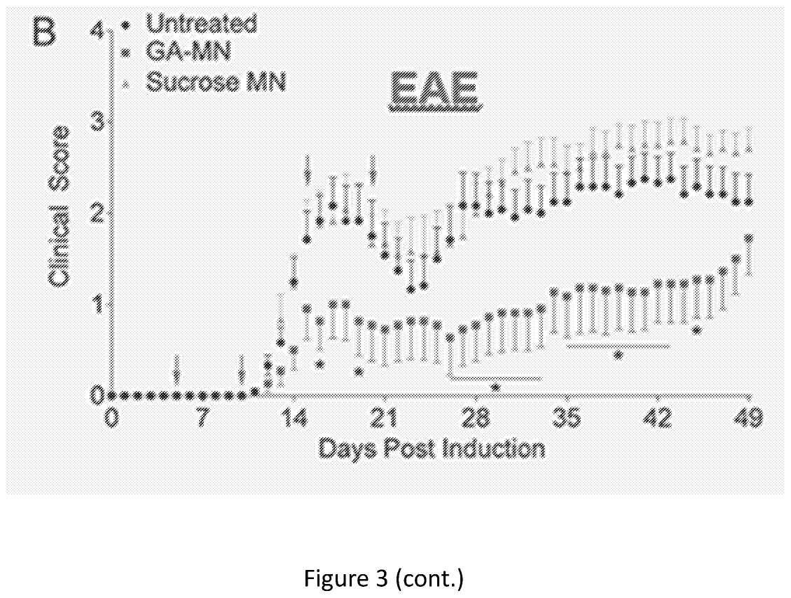

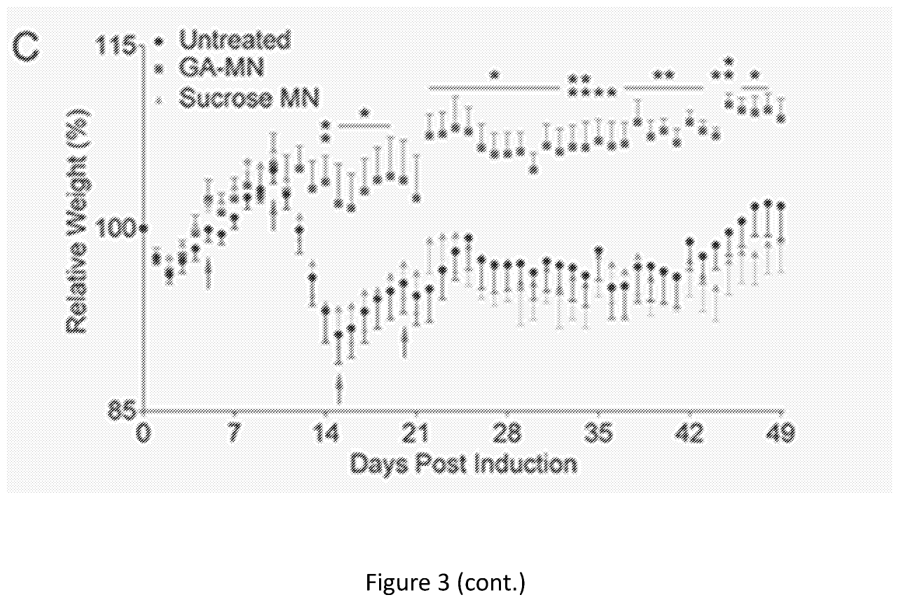

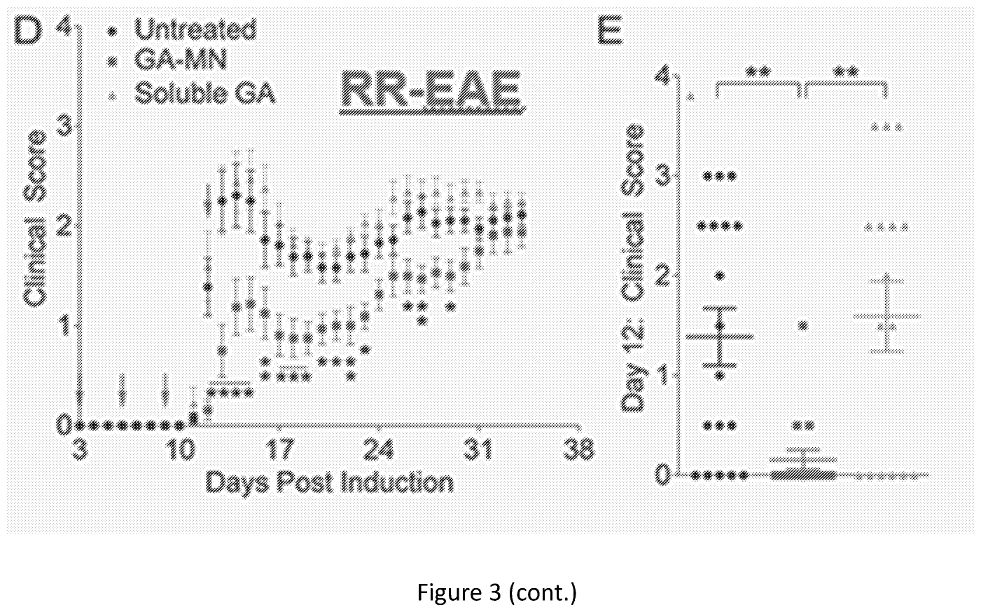

This Example demonstrates that GA-MNs attenuate disease in EAE and relapsing-remitting EAE (RR-EAE). We confirmed the activity and dose-dependence of traditional GA regimens using daily GA injections (i.p.) at 0.2 or 2.0 mg/day beginning at disease onset in EAE (arrows; N=10/group). As discussed above, EAE is a progressive mouse model of MOG-driven inflammation that results in severe paralysis over several weeks; higher clinical scores indicate more severe disease (FIG. 3, panel A). Next we formulated GA-MNs (with sucrose stabilizer) such that a single MN patch contained 0.2 mg—confirmed by dissolution and fluorimetry—to match the ineffective injected GA dose in FIG. 3 panel A. The actual amount of GA delivered from MNs is likely less than the total GA loading due to accumulation on the backing. Mice were then treated at the ear on days 5, 10, 15, and 20 with GA-MNs or control MNs lacking GA (“Sucrose MN”). GA-MNs resulted in significantly decreased EAE scores during treatment that persisted for several weeks after treatment ceased (FIG. 3, panel B). No therapeutic effect was observed with control MNs. Correspondingly, a dramatic drop in weight was observed in mice treated with sucrose MNs, while mice receiving GA-MNs gained weight (FIG. 3, panel C). To analyze this approach in another inflammation model driven by a different myelin peptide—PLP, SJL/J mice were induced with relapsing-remitting EAE (RR-EAE) then treated on days 6, 9, and 12 (N=15/group) using equivalent doses 0.2 mg/treatment (tx) group of GA by MN or soluble i.p. injection. Strikingly, while soluble GA had no effect at this dose, GA-MN significantly improved disease progression (FIG. 3 panel D) and mean score (FIG. 3 panel E) measured at the last treatment day.

### Example 4

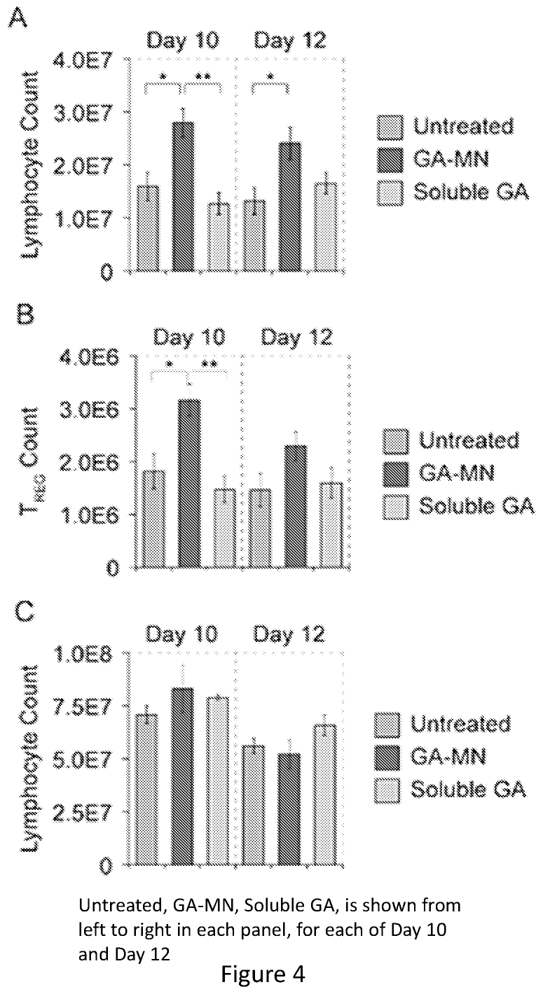

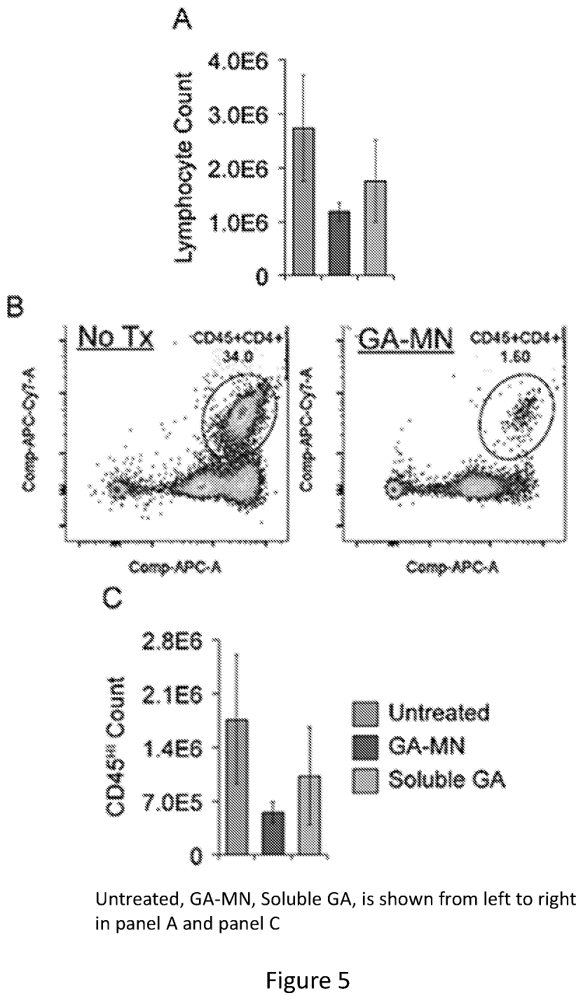

This Example demonstrates that GA-MN increase T_{REG} in cervical LNs and reduce CNS-infiltrating lymphocytes. To analyze this, we analyzed whether GA-MN could increase TREG, and, that because of the decreased clinical scores in FIG. 3, these effects might be evident in cervical LNs. Using analogous regimens to FIG. 3 panel D (RR-EAE), mice were euthanized at Day 10 and Day 12, then cervical LNs and spleen were assessed by flow cytometry. Relative to untreated mice with RR-EAE, GA-MN increased both the number of lymphocytes (FIG. 4 panel A) and the number of T_{REG} (FIG. 4 panel B); these effects were not observed in mice treated with soluble GA. In contrast, no significant differences were observed in the spleen (FIG. 4 panel C). At day 12, we assessed the number of lymphocytes and activated CD4⁺ T cells, as indicated by staining for CD45⁺/CD4⁺ cells. Though the group sizes (N=5) were insufficient for statistical significance, we observed a trend that GA-MN decreased the number of lymphocytes the spinal cord (FIG. 5 panel A), as well as the number of infiltrating CD45⁺CD4⁺ cells (FIG. 5 panels B and C). The data in these Examples indicate GA-MNs deliver GA through the skin of mice at doses that provide efficacy in EAE and RR-EAE. The data also indicate MNs to be more effective than needle injections, and that GA-MA can increase T_{REG} and reduce CNS-infiltrating lymphocytes.

### Example 5

This Example provides additional description of analysis that is contemplated as further embodiments of the disclosure. In one approach, the MNs as described above are applied to a human subject. GA and peptides are released from MNs and are internalized and presented by antigen presenting cells (APCs), and these antigens will be accessible to myelin-reactive T cells. MN delivery will be significantly more potent then free GA or self-antigen. It is expected in human patients that MNs will also offer significant dose sparing, providing efficacy at doses where injected GA does not. Correlating with efficacy, it is expected the GA-MN (and other MN-peptide combinations) will polarize DC function toward tolerance in draining cervical lymph nodes, and for this to translate to expansion of regulatory T cell phenotypes over effector T cells in human patients. Also in lymph nodes, MN cargo is expected to localize to APCs rich in scavenger receptors. Development of tolerogenic microdomains characterized by a high ratio of laminin α4 to laminin α5 can be expected. Corresponding reductions in inflammatory cells in the brain and spinal cord can also be expected.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

1. A method for promoting immune tolerance in an individual comprising intradermal administration of an effective amount of glatiramer acetate to the individual using an array of microneedles, and wherein the immune tolerance is increased relative to a control value for immune tolerance produced by injection of glatiramer acetate.

2. The method of claim 1, wherein a composition comprising the glatiramer acetate is coated on the surface of the microneedles.

3. The method of claim 1, wherein the microneedles are formed by a composition comprising the glatiramer acetate.

4. The method of claim 1, wherein the glatiramer acetate persists in the skin of the individual for a period of at least three days.

5. The method of claim 1, wherein the microneedles comprise ≤2.0 mg of the glatiramer acetate per microneedle array administration.

6. The method of 1, wherein the microneedles comprise not more than 0.2 mg of the glatiramer acetate.

7. The method of claim 1, wherein the immune tolerance comprises:
i) an increase in regulatory T cells (TREGs) in any of: lymph nodes, spleen or the central nervous system of the individual; and/or
ii) a decrease of inflammatory lymphocytes in any of: lymph nodes, spleen or the central nervous system of the individual.

8. The method of claim 7, wherein the increase of TREGS occurs.

9. The method of claim 8, wherein the TREGs are present in cervical lymph nodes of the individual.

10. The method of claim 9, wherein the decrease of inflammatory lymphocytes occurs, and wherein the inflammatory lymphocytes are CD45⁺CD4⁺ T cells.

11. The method of claim 7, wherein the individual has Multiple sclerosis (MS).

12. The method of claim 8, wherein the individual has the MS.

13. The method of claim 9, wherein the individual has the MS.

14. The method of claim 9, wherein the individual has primary-progressive multiple sclerosis (PPMS), relapsing-remitting MS (RRMS), secondary-progressive MS (SPMS), or progressive-relapsing MS (PRMS).

15. The method of claim 14, wherein the individual exhibits an inhibition of onset of paralysis or a reduction in paralysis.

16. The method of claim 14, wherein the array of the microneedles is self-applied by the individual.
